(19) United States
(12) Patent Application Publication
(10) Pub. No.: US 2024/0268946 A1
(43) Pub. Date: Aug. 15, 2024

(54) PROSTHESIS FOR A BYPASS PENDING FUTURE ANASTOMOSIS

(71) Applicant: HOSPICES CIVILS DE LYON, Lyon Cedex (FR)

(72) Inventor: Matthieu ARSICOT, Lyon (FR)

(21) Appl. No.: 18/687,443

(22) PCT Filed: Aug. 30, 2022
(86) PCT No.: PCT/EP2022/074111

§ 371 Date: Feb. 28, 2024

(30) Foreign Application Priority Data
Aug. 31, 2021 (FR) FR2109086

(51) Int. Cl. A61F 2/06; A61F 2/07

(52) U.S. Cl. CPC A61F 2/064; A61F 2/07; A61F 2002/061; A61F 2250/0029

(57) ABSTRACT

The invention relates to a prosthesis (1) for a endobypass pending future anastomosis, having:
a leaktight wall (2) forming a conduit between first and second orifices (25, 26) which are positioned at first and second segments (21, 22) of the leaktight wall;
a first stent (31) encircling the leaktight wall (2) at the first segment;
a second stent (32) encircling the leaktight wall at the second segment;
the leaktight wall having an intermediate segment (23) arranged between the first and second segments and composed of first and second faces (231, 232) extending over respective angular portions;
a third stent (33) integral with the first face of the intermediate segment (23) of the leaktight wall (2), the third stent extending over the entire angular portion of the first face (231), the second face (232) of the intermediate segment (23) being without a stent.

The invention concerns vascular surgery, and in particular the implantation of endobypasses intended for forming a future anastomosis.

For example, a patient suffering from end-stage chronic renal failure may be offered various solutions to alleviate kidney function: hemodialysis, peritoneal dialysis, or kidney transplant.

Kidney transplants are generally performed via an anastomosis on the iliac arterial axis. Documents EP 3 799 831 (INSTITUT FRANçAIS DES SCIENCES ET TECHNOLOGIES DES TRANSP DE LAMENAGEM), and WO 2001/003019 (CORREX INC) deal with anastomosis and more particularly with a T-shaped connector-type prosthesis used as part of an anastomosis. Kidney transplants are limited not only by immunological compatibility, but also by the technical capabilities of the transplant, especially in relation to the condition of the iliac arterial axis. In renal failure, the arteries become calcified and thrombosed. The calcifications prevent arterial clamping and suturing. Arterial thrombosis degrades the permeability of the arterial anastomosis between the donor axis and the transplant.

If the external iliac axis cannot be used for such reasons, the vascular surgeon will perform a parallel bypass of the native artery, which will be the future implantation site for the kidney transplant. The morbidity and mortality associated with this operation is high, with patients generally suffering from multiple pathologies, and may lead to death even before being able to receive a kidney transplant. As a result, such patients are refused kidney transplants.

In particular, the invention aims to solve the problem of morbidity and mortality associated with the arterial preparation for kidney transplant using open bypass surgery.

The invention aims to resolve one or more of these drawbacks. The invention thus relates to a prosthesis for endobypass pending a future anastomosis, as defined in the appended claim 1.

The invention also relates to the variants of the dependent claims. The person skilled in the art will understand that each of the features of the following variants may be combined independently with the above features, without constituting an intermediate generalization.

Further features and advantages of the invention will become clear from the description which follows, by way of indication and by no means limitation, with reference to the appended drawings, wherein:

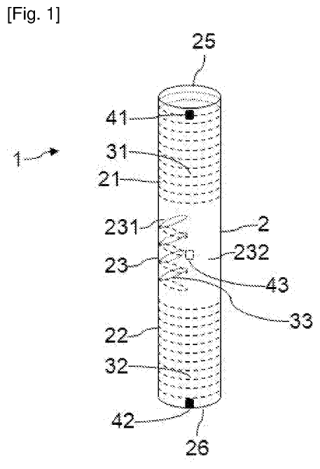

FIG. 1 and

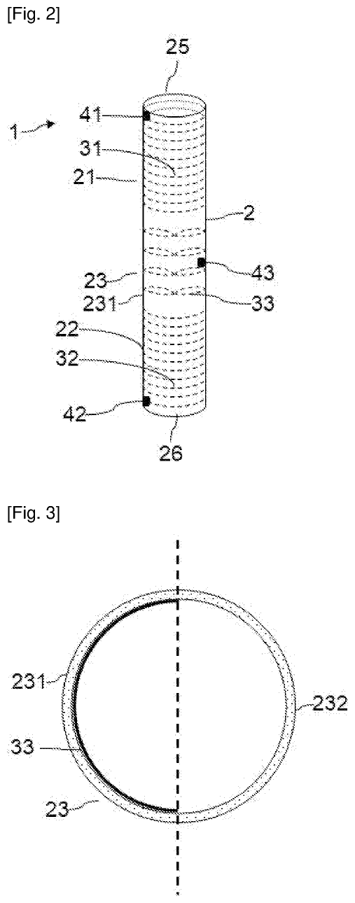

FIG. 2 are perspective views of an example of a prosthesis according to one embodiment of the invention;

FIG. 3 is a cross-sectional view of the prosthesis shown in FIGS. 1 and 2 at the intermediate segment;

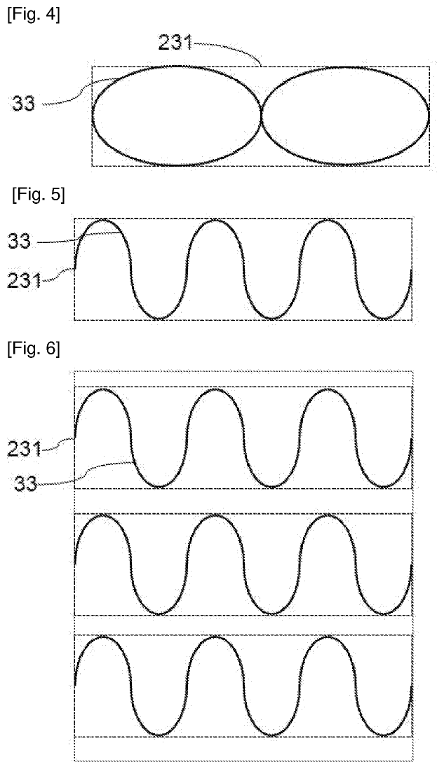

FIG. 4 is an expanded view of one face of an intermediate segment of a prosthesis as shown in FIGS. 1 and 2;

FIG. 5 is an expanded view of a variant of a face of an intermediate segment for a prosthesis according to the invention;

FIG. 6 is an expanded view of another variant of a face of an intermediate segment for a prosthesis according to the invention;

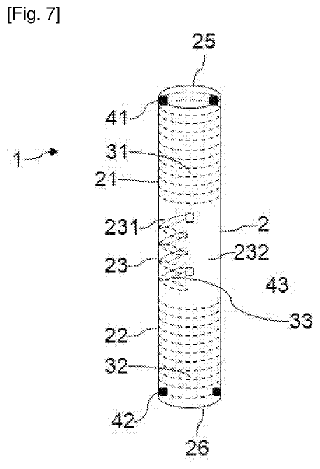

FIG. 7 is a perspective view of an example of a prosthesis according to another example embodiment of the invention;

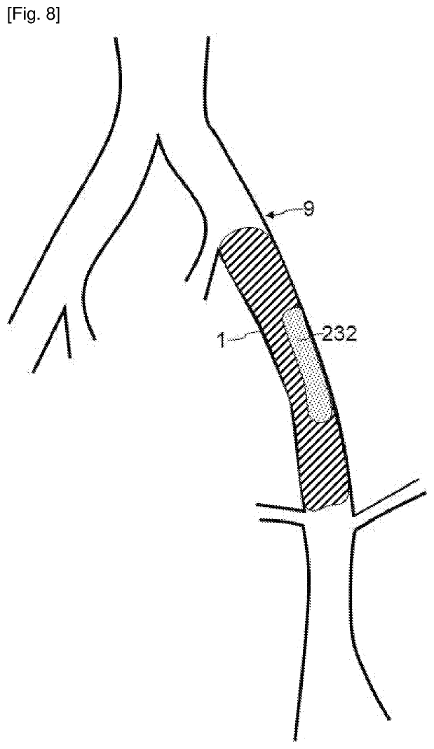

FIG. 8 is a schematic representation of an example of implantation of a prosthesis in the human body.

The endobypass is a known and proven endovascular surgical technique for the percutaneous treatment of arterial occlusions, whether aorto-iliac or femoral. The invention proposes an endoprosthesis for performing such endobypass, by first positioning the endoprosthesis in an arterial axis for future anastomosis.

FIGS. 1 and 2 are perspective views of an example of a prosthesis 1 for endobypass pending future anastomosis, according to one embodiment of the invention. The prosthesis 1 has a leaktight wall 2 forming a conduit between the orifices 25 and 26, positioned at the ends of this conduit. The conduit 2 is designed to guide blood flow between its orifices 25 and 26 through an artery (and avoid an endoleak) that may have pathologies such as calcification or thrombosis.

The leaktight wall 2 has segments 21 and 22 at the openings 25 and 26 respectively. An intermediate segment 23 is positioned between the segments 21 and 22. The intermediate segment 23 forms a continuity between the segments 21 and 22. Advantageously, the leaktight wall 2 has a circular cylindrical shape.

The prosthesis 1 has a stent 31 integral with the segment 21 and encircling the leaktight wall 2. The stent 31 is designed to press the segment 21 against the wall of the artery into which it is inserted in order to ensure blood flow and the absence of endoleaks at the opening 25.

The prosthesis 1 has a stent 32 integral with the segment 22 and encircling the leaktight wall 2. The stent 32 is intended to press the segment 22 against the wall of the artery into which it is inserted to ensure blood flow and the absence of endoleaks at the opening 26.

The stents 21 and 22 here take the form of several stepped rings, but other configurations, in particular continuous stents, may be envisaged in a manner known per se. The stents 21 and 22 may have a plurality of stepped rings separated by pitches of between 2 and 5 mm.

The intermediate segment 23 of the leaktight wall 2 has two opposing faces 231 and 232, extending over respective angular portions about the axis of the leaktight wall 2. The prosthesis 1 also comprises a stent 33 integral with the face 231, an intermediate section 23, and extending over the entire angular portion of the face 231. The face 232 is without a stent. FIG. 3 is a cross-sectional view at the intermediate segment 23 to better distinguish the position of the stent relative to the face 231 and relative to the face 232. The dashed line marks the separation between the faces 231 and 232.

The face 231 is pressed by the stent 33 against the wall of the artery in which the prosthesis 1 is positioned. In this way, the prosthesis 1 ensures blood flow and the absence of endoleaks, pending an anastomosis. The face 232 is without a stent. This face 232 allows for easy opening by a surgeon when preparing an anastomosis, after the prosthesis 1 has been positioned in the artery. The anastomosis may, for example, be used to perform a kidney transplant or arterial bypass. Such prosthesis 1 increases the number of patients eligible for such treatment.

The stent 33 advantageously extends over the entire height of the face 231.

FIG. 4 is an expanded plan view of the face 231, with an example of configuration of the stent 33.

FIG. 5 is an expanded plan view of the face 231, with another example of configuration of the stent 33.

FIG. 6 is an expanded plan view of the face 231, with another example of configuration of the stent 33. The face 231 has a plurality of stepped intermediate segments, each of which has a stent 33 over its entire angular portion. Each stent 33 advantageously extends over the entire height of each intermediate segment. The faces 232 opposite the faces 231 are again without a stent.

Preferably, the height of the segments 21 and 22 respectively extends over at least 20 mm, preferably 40 mm, in order to ensure that the prosthesis 1 is held securely in the artery and to keep the openings 25 and 26 thereof clear.

Preferably, the height of the segment 23 extends over at least 20 mm, preferably 40 mm, to ensure the surgeon has good access to an area for performing an anastomosis.

Preferably, the stent 33 extends advantageously continuously over a height of at least 20 mm, preferably at least 40 mm on the face 231.

Advantageously, to optimize the face 231 pressing against the arterial wall without altering the operation of the practitioner, the angular portion of the face 232 extends over an angle of between 90° and 200° (the angular portion of the face 231 being the complementary angular portion).

Depending on the artery intended for implantation, the leaktight wall 2 has a diameter of between 8 and 35 mm: for example, less than 20 mm for an iliac artery, less than 35 mm for an aortic artery, and less than 12 mm for a femoral artery. Advantageously, the diameter of the leaktight wall 2 is fairly homogeneous, i.e. the ratio between the maximum diameter over its entire height and the minimum diameter over its entire height is less than 1.3.

To facilitate its implantation, the prosthesis 1 advantageously has a leaktight wall with a height of less than 200 mm.

In the example shown in FIG. 1, the prosthesis 1 advantageously has radiolabels to make the operation easier for the practitioner who must perform an anastomosis using this prosthesis 1. The practitioner may thus identify the position of the prosthesis 1 by means of fluoroscopy. The prosthesis 1 has a radiolabel 41 integral with the segment 21, a radiolabel 42 integral with the segment 22 and a radiolabel 43 integral with the segment 23.

In particular, the radiolabels 41 and 42 may be used to identify the ends of the wall 2. The radiolabel 43 allows for identifying the position of the segment 23, comprising the face 232 to be pierced. For example, the radiolabel 43 may be positioned at the boundary between the faces 231 and 232. The radiolabels 41 and 42 are aligned along the axis of the prosthesis 1. The radiolabel 43 is angularly offset from the radiolabels 41 and 42 (typically by 90°) to enable the practitioner to clearly identify the orientation of the prosthesis 1 and the position of the face 232.

FIG. 7 shows another radiolabel configuration. The prosthesis 1 has diametrically opposed radiolabels 41, positioned at the opening 25 and integral with the segment 21. The prosthesis 1 has diametrically opposed radiolabels 42, positioned at the opening 26 and integral with the segment 22. The prosthesis 1 also has radiolabels 43 positioned at the axial ends of the segment 23. The radiolabels 43 are integral with the segment 23. The radiolabels 43 are angularly offset from the radiolabels 41 and 42, typically by 90°.

The stents used may be made of materials known per se, such as nitinol.

FIG. 8 shows a prosthesis 1 present in an artery 9, in anticipation of the subsequent formation of an anastomosis. Here, the anastomosis must be performed in the dashed line area, corresponding to the face 232. The hatched areas correspond to zones on the wall 2 provided with stents.

A prosthesis 1 may be pre-cut to size and positioned percutaneously in the external iliac artery, or in the vicinity of the common femoral artery. Thus, as it is not necessary to surgically open the abdomen for the first stage of preparation for the transplant, the morbidity and mortality are extremely low. This operation may even be performed on an outpatient basis in anatomically favorable cases.

Subsequently, at the time of the transplant, the surgical team benefits from the ease of operating on an abdomen that has not undergone prior surgery, and is therefore without tissue adhesion (apart from other previous operations). Arterial clamping may be performed at the same sites as usual: either proximally at the external iliac artery flush with the internal iliac artery, or distally upstream from the inguinal ligament. Even if the calcifications in the artery cause said artery to tear, the prosthesis 1 inside the artery provides a leaktight seal. Thus, at the time of arterial clamping, the prosthesis 1 has a through-hole on the face 232 without a stent. The arteriotomy is performed on the anterior face of the middle part of the external iliac artery, freeing the endoprothesis from the arterial wall and suturing it to the tissue of the endoprothesis. Once the suture is obtained on the endoprothesis, it is not necessary to close the wall of the native artery.

By extending this technique to patients on the kidney transplant list, the morbidity and mortality associated with arterial preparation procedures may be decreased.

If one draws an analogy with aortic endoprothesis in abdominal aortic aneurysms, the therapeutic attitude of the kidney transplant population could be changed: iliac arteries would be prepared in the first operation by endovascular treatment on an outpatient basis, and open surgery would only occur in the second operation.

The specific endoprothesis may be custom-made prior to the first stage of preparation of the transplant. The diameter of the endoprothesis and its length may then be defined.

The 3 proximal, middle, and distal zones of the endoprothesis remain as defined on a reference base model.

The first surgical stage of endoprothesis placement is performed on an outpatient basis. In the case of percutaneous techniques, the placement of the endoprothesis is performed in the endovascular operating room or the interventional radiology room. The anesthesia technique is not specific: either local anesthesia on a fully alert patient, epidural anesthesia, or general anesthesia may be used, depending on the comorbidities of the patient.

1. A vascular prosthesis (1) for endobypass pending a future anastomosis, characterized in that it has:
a leaktight wall (2) forming a conduit between first and second orifices (25, 26), respectively positioned at first and second segments (21, 22) of the leaktight wall;
a first stent (31) integral with the leaktight wall (2) and encircling the leaktight wall (2) at the first segment;
a second stent (32) integral with the leaktight wall (2) and encircling the leaktight wall at the second segment;
the leaktight wall comprising an intermediate segment (23) between the first and second segments, the intermediate segment (23) has first and second faces (231, 232) extending over respective angular portions;
a third stent (33) integral with the first face of the intermediate segment (23) of the leaktight wall (2), the third stent extending over the entire angular portion of the first face (231), the second face (232) of the intermediate segment (23) being without a stent.

2. The prosthesis (1) for endobypass according to claim 1, wherein the height of the first and second segments (21, 22) extends over at least 20 mm, preferably 40 mm, respectively.

3. The prosthesis (1) for endobypass according to claim 1, wherein the intermediate segment (23) extends to a height of at least 20 mm.

4. The prosthesis (1) for endobypass according to claim 1, wherein said third stent (33) extends over the entire height of the first face.

5. The prosthesis (1) for endobypass according to claim 1, wherein said third stent (33) passes through a cross-section in the intermediate segment (23) at least 4 times.

6. The prosthesis (1) for endobypass according to claim 1, wherein the angular portion of the second face (231) extends over an angle of between 90° and 200°.

7. The prosthesis (1) for endobypass according claim 1, wherein the leaktight wall (2) has a diameter of between 8 and 35 mm.

8. The prosthesis (1) for endobypass according to claim 1, wherein the height of the leaktight wall (2) is less than 200 mm.

9. The prosthesis (1) for endobypass according to claim 1, having a plurality of further intermediate segments between the first and second segments, the further intermediate segments having first and second faces extending over respective angular portions, respective further stents being integral with the respective first faces of said further intermediate segments, said respective further stents each extending over the entire angular portion of the first face of their respective intermediate segment, the respective second face of each further intermediate segment (23) being without a stent.

10. The prosthesis (1) for endobypass according to claim 1, further comprising a radiolabel (43) integral with the first face of the intermediate segment.

11. The prosthesis (1) for endobypass according to claim 1, further comprising a radiolabel (43) integral with the first segment (21).

12. The prosthesis (1) for endobypass according to claim 1, wherein the leaktight wall (2) has a circular cylindrical shape.
